SEQUENCE LISTING (4 sequences)

SEQ ID NO: 1 - DNA, 1833 bp, Homo sapiens
cctcgcagcggtctgcggctccttcccagcccccggcctagctctgcgaacggtgactgcccatccttggccgcaatgagccaccacccgtcgggcctccgggccggcttcagctccacctcataccgccgtaccttcggtccaccgccctcactatcccccggggccttctcctactcgtccagctcccgcttctccagcagccgcctgctgggctccgcgtccccgagctcctcggtgcgcctgggcagcttccgtagcccccgagcgggagcgggcgccctcctgcgcctgccctcggagcgcctcgacttctccatggccgaggccctcaaccaggagttcctggccacgcgcagcaacgagaagcaggagctgcaggagctcaacgaccgcttcgccaacttcatcgagaaggtacgctttctggagcagcagaacgcggccctgcgcggggagctgagccaagcccggggccaggagccggcgcgcgccgaccagctgtgccagcaggagctgcgcgagctgcggcgagagctggagctgttgggccgcgagcgtgaccgggtgcaggtggagcgcgacgggctggcggaggacctggcggcgctcaagcagaggttggaggaggagacgcgcaagcgggaggacgcggagcacaacctcgtgctcttccgcaaggacgtggacgatgccactctgtcccgcctggaactagagcgcaagattgagtctctgatggatgagattgagttcctcaagaagctgcacgaggaggagctgcgagacctgcaggtgagtgtggagagccagcaggtgcagcaggtggaggtggaagccacggtgaagcccgagctgacggcagcgctgagggacatccgcgcgcagtacgagagcatcgccgcgaagaacctgcaggaggcggaggagtggtacaagtccaagtacgcggacctgtccgacgctgccaaccggaaccacgaggccctgcgccaggccaagcaggagatgaacgagtcccgacgccagatccagagtctaacgtgcgaggtggacgggctgcgcggcacgaacgaggcgctgctcaggcagttgagagagctggaggagcagttcgccctggaggcggggggctaccaggcgggcgctgcgcggctcgaggaggagctgcgacagctaaaagaggagatggcgcggcacctgagggagtaccaggagctcctcaacgtcaagatggccctggacatcgagatcgccacctaccgcaagctgctggagggcgaggagagccggatctccgtgcccgtccattcttttgcctccttaaatataaagacgactgtgcctgaggtggagcctccccaggacagccacagccggaagacggttctgatcaagaccattgagacccggaatggggaggtggtgacagagtcccagaaggagcagcgcagtgagctggacaagtcttctgcccacagttactgaaccccttggtccggagccttgactctgccctaggcctgctcaaagcccaaaccctaagaccactcctgaattgtctcctctccctctgcatgtgtctaaaaggtggtaccaggcatccctttcctggcttatggccaagccctacccggccagcagtcgctgggcctctccctgccctgacacttgatgtgacctatgtgcttcccttttcatgtcccgataagaagccaatgatcccccctcaggacaaatctactccagccacgatgagaagtgggtgagccagggtctgagtttcacatttgaaccaaataaaatgctgtcaagagaaaactctccagtgca

SEQ ID NO: 2 - protein, 470 aa, Homo sapiens
MSHHPSGLRAGFSSTSYRRTFGPPPSLSPGAFSYSSSSRFSSSRLLGSASPSSSVRLGSFRSPRAGAGALLRLPSERLDFSMAEALNQEFLATRSNEKQELQELNDRFANFIEKVRFLEQQNAALRGELSQARGQEPARADQLCQQELRELRRELELLGRERDRVQVERDGLAEDLAALKQRLEEETRKREDAEHNLVLFRKDVDDATLSRLELERKIESLMDEIEFLKKLHEEELRDLQVSVESQQVQQVEVEATVKPELTAALRDIRAQYESIAAKNLQEAEEWYKSKYADLSDAANRNHEALRQAKQEMNESRRQIQSLTCEVDGLRGTNEALLRQLRELEEQFALEAGGYQAGAARLEEELRQLKEEMARHLREYQELLNVKMALDIEIATYRKLLEGEESRISVPVHSFASLNIKTTVPEVEPPQDSHSRKTVLIKTIETRNGEVVTESQKEQRSELDKSSAHSY

SEQ ID NO: 3 - DNA, 1774 bp, Mus musculus
cccggcctagttctgccaagcgctgaatgccatcttccgccagcatgagccatcatcactcgtcgggcctgcggtccagcatcagctccacctcgtaccgccggacctttgggccgccgccctcactgtcccccggggctttctcctactcgtccagctctcgcttctccagcagccgcctgctgggctcggggtccccgagctcctcggcgcggctgggcagcttccgtgcccctcgagcgggggcactgcgcttgccctcggagcgcctcgatttctccatggccgaggccctcaaccaagagttcctggccactcggagcaacgagaagcaagagttacaggagctcaacgaccgcttcgccaacttcatcgagaaggtgcgcttcttggagcagcagaacgcagccctgcgcggggagctgagccaggcgcggggccaggagccggcgcgcgccgaccagctttgccagcaggagctgcgcgagctgcggcgcgaactggagctgctgggccgggagcgcgaccgggtgcaggtggagcgggacgggctggcggaggacctagcggcgctcaagcagaggttagaagaagaaacccgcaagcgggaggatgcggagcacaacctggtgctcttccgtaaggacgtggacgacgccactctgtcccgcctagaactggagcgcaagattgagtctctgatggatgaaattgagttcctcaagaagctacacgaagaggaacttcgagacctgcaggtgagcgtagagagccagcaggtgcagcaggtggaggtagaggcaacagtgaagccagagctgacggcggcgctgagggacatccgtgcacagtacgagaacatcgcggcaaagaatctgcaggaggcagaggagtggtataagtcgaaatatgctgacctgtcggacgccgccaaccgcaaccatgaggccctacgccaggccaagcaagagatgaacgagtctcgacgtcagatccagagtctgacgtgcgaggtggatgggctgcgaggcacgaatgaggcgctgctcagacagctgcgggagctggaagagcagttcgccctggaggctggagggtaccaagcaggcgcagcacggctggaggaagagcttcgacagctgaaggaagagatggcgaggcacctgcgagagtaccaggagctccttaacgtcaagatggccctggacatcgagatagccacctacaggaagctactggaaggggaggagagccggatctcagtgccggttcattcctttgcctctctaagtttaaagacgactgtgcctgagatggagcctctccaggatagccacagcaagaagatggttctgatcaggacaattgagacccgggatggggagaaggtggtgacagagtcccagaaggaacagcacagtgacctggacaagtcttctatccacagctactgaggcctcagccagagctctgaccctgatctcagcctattcctaagcttggccctccccagcaccagtctgtatccagtcctgctctgcatgccacagcccctgcctgccaagcaagcgccagcctgatccagaggctgggcctcttcccgatgcataggtgtgacccatagctttcttgtccttgtaagaggtggataaagatacccaggacagcaaatctactcaagaatgaccccatggtctgtggggtgggccagagtgtgggactcactctgagtcaaataaaactgctactgagagaaaaaaaaaaaaaaaaaaaa

SEQ ID NO: 4 - protein, 475 aa, Mus musculus
MPSSASMSHHHSSGLRSSISSTSYRRTFGPPPSLSPGAFSYSSSSRFSSSRLLGSGSPSSSARLGSFRAPRAGALRLPSERLDFSMAEALNQEFLATRSNEKQELQELNDRFANFIEKVRFLEQQNAALRGELSQARGQEPARADQLCQQELRELRRELELLGRERDRVQVERDGLAEDLAALKQRLEEETRKREDAEHNLVLFRKDVDDATLSRLELERKIESLMDEIEFLKKLHEEELRDLQVSVESQQVQQVEVEATVKPELTAALRDIRAQYENIAAKNLQEAEEWYKSKYADLSDAANRNHEALRQAKQEMNESRRQIQSLTCEVDGLRGTNEALLRQLRELEEQFALEAGGYQAGAARLEEELRQLKEEMARHLREYQELLNVKMALDIEIATYRKLLEGEESRISVPVHSFASLSLKTTVPEMEPLQDSHSKKMVLIRTIETRDGEKVVTESQKEQHSDLDKSSIHSY

(19) United States
(12) Patent Application Publication
(10) Pub. No.: US 2013/0023484 A1
(43) Pub. Date: Jan. 24, 2013

(54) PERIPHERIN-SPECIFIC AUTOANTIBODIES AS A MARKER FOR NEUROLOGICAL AND ENDOCRINOLOGICAL DISEASE

(71) Applicants: Vanda A. Lennon, Rochester, MN (US); Jayne Chamberlain, Gwynedd (GB); Thomas J. Kryzer, Mantorville, MN (US); Sean J. Pittock, Rochester, MN (US); Anna Maria Oprescu, Ridgewood, NY (US)

(72) Inventors: Vanda A. Lennon, Rochester, MN (US); Jayne Chamberlain, Gwynedd (GB); Thomas J. Kryzer, Mantorville, MN (US); Sean J. Pittock, Rochester, MN (US); Anna Maria Oprescu, Ridgewood, NY (US)

(73) Assignee: MAYO FOUNDATION FOR MEDICAL EDUCATION AND RESEARCH, Rochester, MN (US)

(21) Appl. No.: 13/519,089

(22) PCT Filed: Jan. 4, 2011
(86) PCT No.: PCT/US11/20114

§ 371 Date: Jun. 25, 2012

Related U.S. Application Data
(60) Provisional application No. 61/292,031, filed on Jan. 4, 2010.

(51) Int. Cl. G01N 33/566; A61M 1/36; A61P 37/06; A61K 38/17; G01N 33/577

(52) U.S. Cl. 514/20.9; 436/501; 435/7.1; 604/522

(57) ABSTRACT

The present invention provides for methods and materials for detecting a peripherin-specific autoantibody, which can be associated with neurological and endocrinological disease.

### CROSS-REFERENCE TO RELATED APPLICATIONS

This application claims the benefit of priority to U.S. Provisional Application Ser. No. 61/292,031, filed on Jan. 4, 2010. The disclosure of the prior application is considered part of (and is incorporated by reference in) the disclosure of this application.

### FEDERALLY SPONSORED RESEARCH OR DEVELOPMENT

This invention was made with government support under Grant Nos. DK71209 and DK68055 awarded by National Institutes of Health. The government has certain rights in the invention.

### TECHNICAL FIELD

This disclosure relates generally to neurological and endocrinological disease, and more particularly, to the identification of peripherin-specific antoantibodies as a marker for such neurological and endocrinological disease.

### BACKGROUND

Neural-restricted autoantibodies are emerging as serum biomarkers of acquired neurological disorders, both idiopathic and paraneoplastic. Autoantigens identified to date are expressed in neurons, glia or skeletal muscle. Plasma membrane autoantigens are potential targets of pathogenic IgG and are exemplified by channel proteins. These include neuronal voltage-gated potassium channels and NMDA and AMPA receptors in encephalopathies, astrocytic water channels in neuromyelitis optica, muscle acetylcholine receptors [AChR] in myasthenia gravis and neuronal AChR in autoimmune autonomic ganglionopathies. Antibodies recognizing intracellular autoantigens are not pathogenic to intact cells. These include IgGs reactive with neuronal and glial cytoplasmic and nuclear proteins such as glutamic acid decarboxylase-65 (GAD65) in stiff-man syndrome, cerebellitis and encephalomyelopathy, and anti-neuronal and glial nuclear autoantibodies in multifocal paraneoplastic disorders.

### SUMMARY

Peripherin-specific autoantibodies have been identified as a serum biomarker in individuals with autoimmune neurological disorders (particularly targeting the peripheral autonomic and somatic nervous system, spinal cord and optic nerves) often in the context of endocrinological disease, and sometimes with cancer. The detection of peripherin-specific autoantibodies can be used to support the diagnosis of an individual with autoimmune neurological and endocrinological disease.

In one aspect, a method of detecting the presence or absence of a peripherin-specific autoantibody in a biological sample from an individual is provided. Such a method generally includes the steps of contacting the biological sample with a peripherin polypeptide or fragment thereof; and detecting the presence or absence of binding of the peripherin polypeptide or fragment thereof to the peripherin-specific autoantibody in the biological sample. Typically, the presence of the peripherin-specific autoantibody in the biological sample is associated with dysautonomia or small fiber neuropathy, endocrinopathy, myelopathy, visual impairment, or other neurological manifestations in the individual. In certain embodiments, the peripherin polypeptide or fragment thereof is in a cell lysate; in other embodiments, the peripherin polypeptide is in a solid tissue selected from the group consisting of brain (e.g., hindbrain), kidney, stomach or other tissues containing peripheral nerve elements.

Representative examples of dysautonomia include gastrointestinal (GI) dysmotility, abnormal sudomotor function, abnormal cardiovagal function, or abnormal adrenergic function. Representative examples of endocrinopathy include diabetes, thyroid disorders, or premature menopause. Representative examples of neurological manifestations include transverse myelitis, non-specified myelopathy, optic neuropathy, encephalitis, lumbosacral plexopathy, sensorimotor neuropathy, cerebellar ataxia or myasthenia gravis. In some embodiments, the biological sample is selected from the group consisting of serum, plasma, cerebrospinal fluid, and blood.

In another aspect, an article of manufacture is provided. Such an article of manufacture generally includes a peripherin polypeptide or fragment thereof and instructions for using the peripherin polypeptide to detect an anti-peripherin autoantibody in an individual. Typically, the article of manufacture is used to diagnose the presence or absence of a peripherin-associated autoimmune disease in the individual. In certain embodiments, an article of manufacture further includes a monoclonal antibody having specific binding affinity for a peripherin polypeptide or fragment thereof.

In yet another aspect, a method of treating an individual having a peripherin-associated autoimmune disease is provided. Such a method typically includes withdrawing a body fluid from the individual, wherein the body fluid contains one or more autoantibodies that bind to peripherin; removing a substantial portion of the autoantibodies from the body fluid; and returning the body fluid to the subject.

In still another aspect, a method of treating an individual having a peripherin-associated autoimmune disease is provided. Such a method typically includes administering a peripherin polypeptide to the individual. In some instances, administration is by a method selected from the group consisting of orally, intravenously, and parenterally.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods and examples are illustrative only and not intended to be limiting. All publications, patent applications, patents and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 is the nucleotide (SEQ ID NO:1) and amino acid (SEQ ID NO:2) sequence of human peripherin.

FIG. 2 is the nucleotide (SEQ ID NO:3) and amino acid (SEQ ID NO:4) sequence of mouse peripherin.

FIG. 3 are photographs demonstrating that a novel IgG autoantibody binds to neural elements in sections of mouse stomach and kidney and to discrete fiber tracts in mid-hind brain. Bound IgG was visualized using fluorescein-conjugated anti-human IgG. The characteristic staining pattern of this autoantibody is prominent in myenteric ganglia, nerve fibers and nerve trunks in the enteric nervous system (upper panel), sympathetic nerve trunks and fibers adjacent to arterioles in the gastric submucosa and kidney (center panel) and discrete nerve bundles in the mid-hind brain (lower panel). G: ganglion; NT: nerve trunk; NF: nerve fibers; SN: sympathetic nerve.

FIG. 4 are photographs demonstrating that the antigen associated with the novel autoantibodies described herein is restricted to neurons. Differentiated cell lines used as substrates: pheochromocytoma (PC12; left panels), astrocytes (CG4; middle panels) and skeletal muscle (L6; right panels). Informative IgG probes (from top) included: Neurofilament (neuronal), GFAP (glial) and striational (skeletal muscle sarcomere), and an individual patient serum. Only the neuronal cell line was immunoreactive with the individual patient serum.

FIG. 5 are results demonstrating that the novel IgG autoantibody binds to a protein having a Mr of 55-60 kDa that is confined to the neuronal cytoskeleton. (A) Using PC12 lysate as a source of antigen, proteins were separated, immunoblotted, and probed with individual patient or normal human sera. A common band (˜55 kDa by reference to molecular weight standards) was revealed by IgG in individuals' sera (lanes 1-4), but not by IgG in control human serum (lanes 5-6). To verify specificity, individual IgG was affinity-purified on the putative antigenic band and a control band. Eluates from the putative antigenic band (B) and control band (C) were re-applied to the composite mouse tissue substrate slide, and compared to the original immunostaining pattern of whole individual serum IgG on enteric nerve fibers (D). An identical staining pattern was observed in eluates from the putative antigenic band. To determine the subcellular distribution of the antigen, PC12 lysates were fractionated by differential detergent extraction. Proteins in each fraction were resolved electrophoretically, transblotted, and probed with individual sera (E). IgG bound exclusively to a protein in the cytoskeletal fraction.

FIG. 6 are results that identify peripherin as the antigen. Duplicate preparations of the cytoskeletal fraction of PC12 cells were separated by sequential gel electrophoresis and isoelectric focusing. One gel was silver stained (A), and the replica was transblotted and probed with individual serum (B). Unique peptides yielded by in-gel digestion from the silver stained gel of the common spots (numbered 1-4) identified the antigen to be peripherin.

FIG. 7 are photographs that demonstrate that individual IgG colocalizes with peripherin immunoreactivity in brain and endocrine organs. Tissues (brain [A-C], thyroid [D-F], pancreas [G-I], ovary [J-L] and liver [M-O]) were harvested from a 6-8 week old female mouse, cryosections (8 μm) were cut and stained with rabbit anti-peripherin-IgG (left columns) and individual patient IgG (center columns). All merged images (right column) show nuclear DAPI staining (blue) except for pancreas, where endocrine islet cells (I) were identified by IgG specific for the β-cell transcription factor PDX-1 (pseudo-colored purple).

### DETAILED DESCRIPTION

A specific IgG autoantibody marker has been identified in serum of individuals presenting with dysautonomia (e.g., gastrointestinal (GI) dysmotility, abnormal sudomotor function, abnormal cardiovagal function or abnormal adrenergic function), endocrinopathy (e.g., diabetes, thyroid disorders, or premature menopause) and other neurological disorders (e.g., particularly myelopathies, optic and other cranial neuropathies, cerebellar ataxia and sensory-predominant neuropathies but also lumbosacral plexopathy, encephalitis, or myasthenia gravis). Importantly, the clinical presentation of patients with myelopathy or visual impairment mimics that of multiple sclerosis. The target of this novel autoantibody has been identified as peripherin, a type III neuronal intermediate filament protein that forms networks, either alone or complexed with other neurofilament proteins. It is attributed a role in neuron development and repair, and is distributed widely in the peripheral nervous system. In the central nervous system, it is restricted to regions that project to the periphery.

Thus, this disclosure provides for methods of detecting peripherin-specific autoantibodies in an individual that presents with dysautonomia, myelopathy, vision impairment, endocrinopathy, sensory-predominant neuropathies or other neurological disorders. In a diabetes environment, the presence of peripherin-specific autoantibodies may be used to evaluate the individual's risk for developing neurological complications (e.g., small fiber neuropathy). In a multiple sclerosis or multiple sclerosis-like setting, the presence of peripherin-specific autoantibodies may be used to evaluate the individual's potential to benefit from immunosuppressant therapies.

### Peripherin Polypeptides and Anti-Peripherin Antibodies

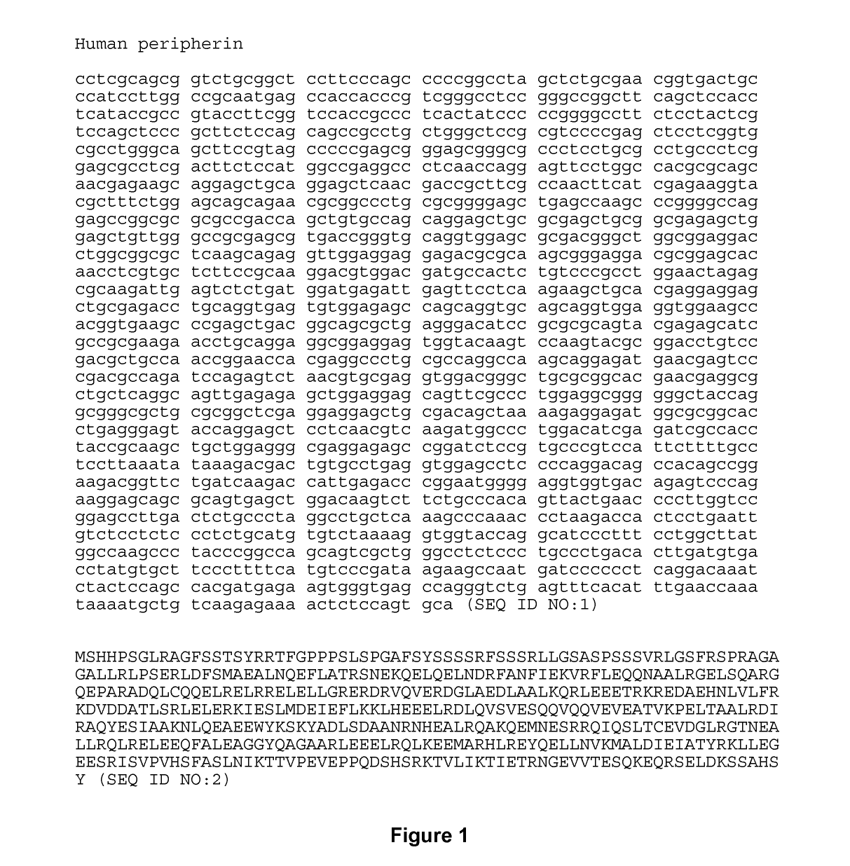

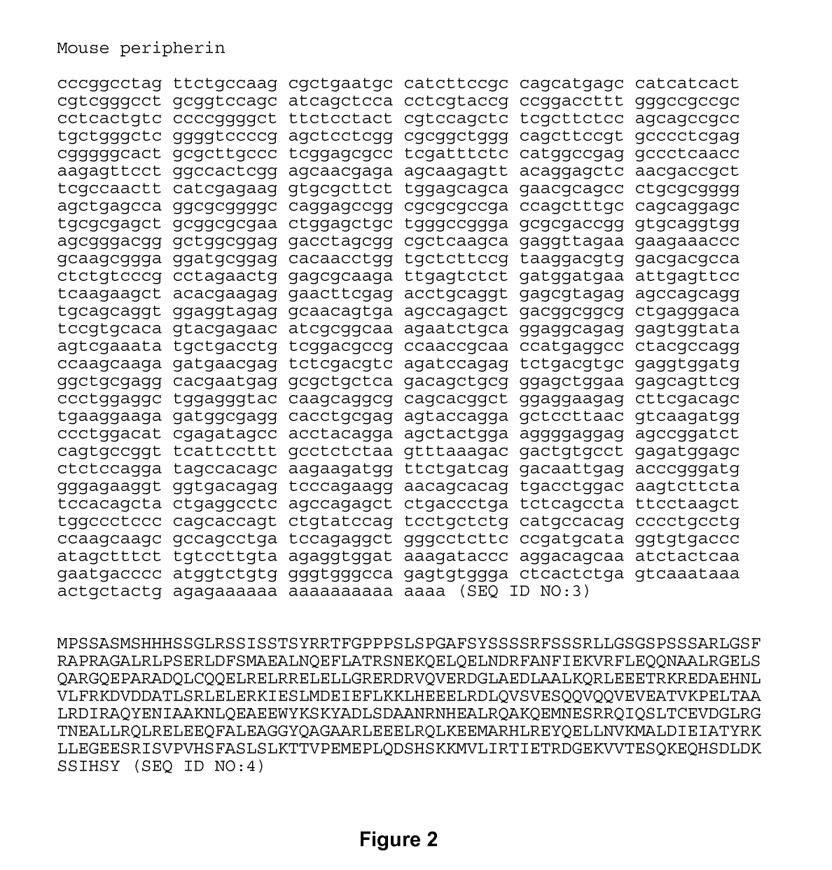

Peripherin polypeptides can be used in the methods described herein (e.g., detecting a peripherin-specific autoantibody). Examples of peripherin polypeptide sequences (and the nucleic acids encoding such polypeptides) can be found in GenBank Accession Nos. BC046291; NM_{—}001001235; NM_{—}006262; NM_{—}012633; BC100656; NM_{—}001087060; and NM_{—}131054. Additional peripherin sequences can be found, for example, in public databases. A representative human peripherin sequence is shown in FIG. 1 (SEQ ID NOs: 1 and 2; DNA and protein, respectively), and a representative mouse peripherin sequence is shown in FIG. 2 (SEQ ID NOs: 3 and 4; DNA and protein, respectively).

Peripherin polypeptides, like other intermediate filaments, are insoluble. However, peripherin polypeptides are well-studied, and there are many reports in the literature that describe methods of working with peripherin. See, for example, Portier et al. (1983-4, Dev. Neurosci., 6(6):335-44); Landon et al. (2000, Biol. Cell., 92(6):397-407); McLean et al. (2008, J. Neurochem., 104(6):1663-73); Puertas et al. (2007, J. Immun., 178(10):6533-39); and Aletta et al. (1989, J. Biol. Chem., 264(8):4619-27). In addition, those skilled in the art are aware of methods that can be used to increase the solubility of a polypeptide. See, for example, Trimpin & Brizzard, 2009, Bio Techniques, 46(6):409-19.

Peripherin polypeptides may be obtained from human, mouse or other mammalian neuronal tissue, neuronal cell lines, or transfected cells (e.g., mammalian, E. coli or yeast) expressing a recombinant peripherin nucleic acid, or the peripherin polypeptide may be synthetic. Polypeptides can be purified. A “purified” polypeptide refers to a polypeptide that constitutes the major component in a mixture of components, e.g., 30% or more, 40% or more, 50% or more, 60% or more, 70% or more, 80% or more, 90% or more, 95% or more, or 99% or more by weight. Polypeptides may be purified by methods including affinity chromatography or immunosorbant affinity column. Such methods can be modified by those skilled in the art to increase the solubility of the polypeptide, and purified polypeptides can be examined for their immunogenicity using routine methods.

Given a peripherin polypeptide sequence (see, for example, SEQ ID NOs: 2 and 4), virtually any polypeptide fragment can be generated by known means (e.g., proteolytic cleavage of a polypeptide or chemical synthesis). It would be understood by those skilled in the art that a fragment of peripherin may have a different solubility than that of the full-length peripherin polypeptide. Fragments of a peripherin polypeptide can contain one or more epitopic sites. Epitopic sites within peripherin polypeptides that are pertinent to T-cell activation and suppression (e.g., MHC-I and MHC-II binding epitopes) can be determined by direct investigation, or by using computer algorithms. See, for example, Parker et al. (J. Immunol., 152:163 (1994)) and Southwood et al. (J. Immunol., 160:3363 (1998)). Usually, it is desirable that the epitopic site be antigenically distinct from other closely related antigens (e.g., other members of a family of polypeptides).

Peripherin polypeptides or fragments thereof may be used with or without modification for the detection of peripherin-specific antibodies such as peripherin-specific autoantibodies. Polypeptides can be labeled by either covalently or non-covalently combining the polypeptide with a second substance that provides for detectable signal. A wide variety of labels and conjugation techniques are known in the art and are reported extensively in both the scientific and patent literature. Some of the labels include radioisotopes, enzymes, substrates, cofactors, inhibitors, fluorescers, chemiluminescers, magnetic particles, and the like.

Peripherin polypeptides or fragments thereof can be used in various immunological techniques to detect peripherin-specific antibodies. For example, peripherin polypeptides can be used in an immunoassay to detect peripherin-specific autoantibodies in a biological sample. Peripherin polypeptides used in an immunoassay can be in a cell lysate (e.g., a whole cell lysate or a cell fraction), or purified peripherin polypeptides or fragments thereof can be used provided at least one antigenic site recognized by peripherin-specific antibodies (e.g., peripherin-specific autoantibodies) remains available for binding. Depending on the nature of the sample, either or both immunoassays and immunocytochemical staining techniques may be used. Enzyme-linked immunosorbent assays (ELISA), Western blot, and radioimmunoassays are methods used in the art, and can be used as described herein to detect the presence of peripherin-specific autoantibodies in a biological sample.

A “biological sample,” as used herein, is generally a sample from an individual. Non-limiting examples of biological samples include blood, serum, plasma, or cerebrospinal fluid. Additionally, solid tissues, for example, spinal cord or brain biopsies may be used.

Further provided in this disclosure are articles of manufacture (e.g., kits) containing one or more peripherin polypeptides or fragments thereof. Peripherin polypeptides or fragments thereof that are included in an article of manufacture as described herein can be provided within a cell, in a solution in which they are soluble, or the peripherin polypeptides or fragments thereof can be provided in a lyophilized form. In certain embodiments, an article of manufacture as described herein also can include one or more compounds for increasing the solubility of a polypeptide (e.g., a solubilizing agent). The kit may further include a second substance that, for example, provides for a detectable signal. In addition, a kit can include directions for using the peripherin polypeptides and/or directions for practicing a method described herein (i.e., detecting peripherin-specific autoantibodies in a biological sample).

The present disclosure also provides for methods of detecting peripherin polypeptides. Detection of a polypeptide is typically performed using an antibody, referred to herein as an anti-peripherin antibody to distinguish such animal- or recombinantly-generated antibodies from peripherin-specific autoantibodies produced by an individual's immune system. This disclosure also provides for an antibody, including a monoclonal antibody, with specific binding affinity for peripherin polypeptides or antigenic fragments thereof.

Peripherin polypeptides as described herein can be used to produce monoclonal or polyclonal anti-peripherin antibodies having specific binding affinity for the peripherin polypeptide. Such antibodies can be produced using techniques known to those of ordinary skill in this art. As used herein, anti-peripherin antibodies having “specific binding affinity” for peripherin polypeptides or fragments thereof are defined as those antibodies that preferentially bind peripherin polypeptides or fragments thereof, but that do not bind or have very little affinity for non-peripherin polypeptides. While the peripherin-specific autoantibodies described herein are IgG antibodies, recombinantly produced “anti-peripherin antibody” can be whole antibodies of any class (e.g., IgG, IgA, IgM), portions or fragments of whole antibodies (e.g., Fab or (Fab)₂ fragments) having the desired specific binding affinity, an engineered single chain Fv molecule, or a chimeric molecule, e.g., an antibody that contains the binding specificity of one antibody (e.g., of murine origin) and the remaining portions of another antibody (e.g., of human origin).

Anti-peripherin antibodies may be used with or without modification for the detection of peripherin polypeptides. Anti-peripherin antibodies can be labeled either directly or indirectly, and a wide variety of labels, including radioisotopes, enzymes, substrates, cofactors, inhibitors, fluorescers, chemiluminescers and magnetic particles, and conjugation techniques are known and are reported extensively in both the scientific and patent literature. Also provided by this disclosure is an anti-peripherin antibody having specific binding affinity for peripherin polypeptides conjugated to an imaging agent. Suitable imaging agents include, but are not limited to, radioisotopes, such as ³²P, ⁹⁹Tc, ¹¹¹In and ¹³¹I.

Anti-peripherin antibodies as described herein can be used in various immunological techniques for detecting peripherin polypeptides. The use of antibodies in protein binding assays is well established. Depending on the nature of the sample, immunoassays (e.g., radioimmunoassays) and/or immunohistochemical/immunocytochemical staining techniques may be used. Liquid phase immunoassays (e.g., competitive inhibition radioimmunoassays) or solid phase immunoassays (e.g., antigen-capture or Western blot analysis) can also be used to detect peripherin polypeptides. Additionally, enzyme-linked immunosorbent assays (ELISA) are routinely practiced in the art, and may be used for detecting the presence of peripherin polypeptides.

Further provided by this disclosure is a kit containing anti-peripherin antibodies having binding affinity for peripherin polypeptides or fragments thereof. The kit may also include peripherin polypeptides or fragments thereof to be used as binding controls or to generate a standardized quantitative curve. The kit may further include a second substance that provides for detectable label. A kit typically includes directions for using an anti-peripherin antibody (e.g., for detecting or purifying peripherin polypeptides).

### Peripherin Nucleic Acids and Constructs

As used herein, nucleic acid (e.g., peripherin nucleic acid) refers to RNA or DNA. As used herein with respect to nucleic acids, “isolated” refers to (i) a nucleic acid sequence encoding part or all of peripherin polypeptide, but free of coding sequences that normally flank one or both sides of the nucleic acid sequences encoding peripherin in the genome; or (ii) a nucleic acid incorporated into a vector or into the genomic DNA of an organism such that the resulting molecule is not identical to any naturally-occurring vector or genomic DNA.

Representative peripherin nucleic acids are shown in FIGS. 1 and 2 (SEQ ID NO:1 and 3), as well as in the GenBank Accession Nos. provided herein. Peripherin nucleic acids also can include fragments of peripherin nucleic acid sequences. As used herein, fragments refer to nucleic acids or polypeptides corresponding to less than an entire peripherin sequence. Nucleic acid fragments may include those fragments of about 10 to 50 nucleotides in length, fragments of about 20 to 100 nucleotides in length, or fragments that are 100 to several hundred nucleotides in length. Such fragments may, for example, encode a peripherin polypeptide fragment, or have utility as hybridization probes or amplification primers.

Given a peripherin nucleotide sequence (see, for example, SEQ ID NOs: 1 and 3), virtually any nucleic acid fragment can be generated by known means (e.g., restriction enzyme digestion, the polymerase chain reaction) and, if so desired, expressed to produce the corresponding polypeptide fragment. Various restriction enzyme sites within a peripherin nucleic acid sequence define positions that, in various combinations, can be used to generate useful nucleic acid fragments.

A peripherin nucleic acid or nucleic acid fragment may have a sequence that deviates from a wild type peripherin sequence (e.g., SEQ ID NOs:1 and 3), sometimes referred to as a variant sequence. For example, a variant nucleic acid sequence can have at least 80% sequence identity to a wild type peripherin sequence (e.g., SEQ ID NOs: 1 and 3). In some embodiments, the variant nucleic acid sequence can have at least 85% sequence identity, 90% sequence identity, 95% sequence identity, or at least 99% sequence identity to a wild type peripherin sequence (e.g., SEQ ID NOs: 1 and 3). Variant nucleic acid sequences can be designed that encode variant peripherin polypeptides or fragments thereof that, for example, exhibit increased solubility compared to the wild type polypeptides or fragments thereof.

Percent sequence identity is calculated by determining the number of matched positions in aligned nucleic acid or polypeptide sequences, dividing the number of matched positions by the total number of aligned nucleotides or amino acids, respectively, and multiplying by 100. A matched position refers to a position in which identical nucleotides or amino acids occur at the same position in aligned sequences. The total number of aligned nucleotides or amino acids refers to the minimum number of peripherin nucleotides or amino acids that are necessary to align the second sequence, and does not include alignment (e.g., forced alignment) with non-peripherin sequences, such as those fused to peripherin. The total number of aligned nucleotides or amino acids may correspond to the entire peripherin sequence or may correspond to fragments of the full-length peripherin sequence as defined herein.

Sequences can be aligned using the algorithm described by Altschul et al. (1997, Nucleic Acids Res., 25:3389-3402) as incorporated into BLAST (basic local alignment search tool) programs, available at ncbi.nlm.nih.gov on the World Wide Web. BLAST searches or alignments can be performed to determine percent sequence identity between a peripherin nucleic acid molecule and any other sequence or portion thereof using the Altschul et al. algorithm. BLASTN is the program used to align and compare the identity between nucleic acid sequences, while BLASTP is the program used to align and compare the identity between amino acid sequences. When utilizing BLAST programs to calculate the percent identity between a peripherin sequence and another sequence, the default parameters of the respective programs are used.

A nucleic acid encoding a peripherin polypeptide may be obtained from, for example, a cDNA library made from a human or rat cell line, or can be obtained by other means, including, but not limited to, the polymerase chain reaction (PCR). PCR refers to a procedure or technique in which target nucleic acids are amplified. PCR can be used to amplify specific sequences from DNA as well as RNA, including sequences from total genomic DNA or total cellular RNA. Various PCR methods are described, for example, in PCR Primer: A Laboratory Manual, Dieffenbach & Dveksler, Eds., Cold Spring Harbor Laboratory Press, 1995. Generally, sequence information from the ends of the region of interest or beyond is employed to design oligonucleotide primers that are identical or similar in sequence to opposite strands of the template to be amplified.

Peripherin nucleic acids can be detected by methods such as Southern or Northern blot analysis (i.e., hybridization), PCR, or in situ hybridization analysis. Hybridization between nucleic acid molecules is discussed in detail in Sambrook et al. (1989, Molecular Cloning: A Laboratory Manual, 2^{nd} Ed., Cold Spring Harbor Laboratory Press, Cold Spring Harbor, N.Y.; Sections 7.37-7.57, 9.47-9.57, 11.7-11.8, and 11.45-11.57).

For oligonucleotide probes less than about 100 nucleotides, Sambrook et al. discloses suitable Southern blot conditions in Sections 11.45-11.46. The Tm between a sequence that is less than 100 nucleotides in length and a second sequence can be calculated using the formula provided in Section 11.46. Sambrook et al. additionally discloses prehybridization and hybridization conditions for a Southern blot that uses oligonucleotide probes greater than about 100 nucleotides (see Sections 9.47-9.52). Hybridizations with an oligonucleotide greater than 100 nucleotides generally are performed 15-25° C. below the Tm. The Tm between a sequence greater than 100 nucleotides in length and a second sequence can be calculated using the formula provided in Sections 9.50-9.51 of Sambrook et al. Additionally, Sambrook et al. recommends the conditions indicated in Section 9.54 for washing a Southern blot that has been probed with an oligonucleotide greater than about 100 nucleotides.

The conditions under which membranes containing nucleic acids are prehybridized and hybridized, as well as the conditions under which membranes containing nucleic acids are washed to remove excess and non-specifically bound probe can play a significant role in the stringency of the hybridization. Such hybridizations can be performed, where appropriate, under moderate or high stringency conditions. Such conditions are described, for example, in Sambrook et al. section 11.45-11.46. For example, washing conditions can be made more stringent by decreasing the salt concentration in the wash solutions and/or by increasing the temperature at which the washes are performed. In addition, interpreting the amount of hybridization can be affected, for example, by the specific activity of the labeled oligonucleotide probe, by the number of probe-binding sites on the template nucleic acid to which the probe has hybridized, and by the amount of exposure of an autoradiograph or other detection medium.

It will be readily appreciated by those of ordinary skill in the art that although any number of hybridization and washing conditions can be used to examine hybridization of a probe nucleic acid molecule to immobilized target nucleic acids, it is more important to examine hybridization of a probe to target nucleic acids under identical hybridization, washing, and exposure conditions. Preferably, the target nucleic acids are on the same membrane.

A nucleic acid molecule is deemed to hybridize to a first target nucleic acid but not to a second target nucleic acid if hybridization to the first nucleic acid is at least 5-fold (e.g., at least 6-fold, 7-fold, 8-fold, 9-fold, 10-fold, 20-fold, 50-fold, or 100-fold) greater than hybridization to the second nucleic acid. The amount of hybridization can be quantitated directly on a membrane or from an autoradiograph using, for example, a Phosphorlmager or a Densitometer (Molecular Dynamics, Sunnyvale, Calif.).

Also provided herein are vectors containing a peripherin nucleic acid (see, for example, SEQ ID NOs: 1 and 3) or the complements thereof, peripherin nucleic acid fragments or the complements thereof, and those nucleic acids having at least 80% sequence identity to a peripherin nucleic acid or fragments generated therefrom (or the complements thereof). Cloning vectors are commercially available and used routinely by those of ordinary skill. Vectors may additionally comprise elements necessary for expression operably linked to a peripherin nucleic acid sequence. “Elements necessary for expression” include promoter sequences, and additionally may include regulatory elements, such as enhancer sequences, response elements or inducible elements that modulate expression of a peripherin nucleic acid sequence. As used herein, “operably linked” refers to positioning of a promoter and/or other regulatory element(s) in a construct relative to a peripherin nucleic acid sequences in such a way as to direct or regulate expression of the peripherin nucleic acid. Such constructs are commercially available (e.g., expression vectors) and/or produced by recombinant DNA technology methods routine in the art. The choice of expression systems depends upon several factors, including, but not limited to, replication efficiency, selectability, inducibility, targeting, the level of expression desired, ease of recovery and the ability of the host to perform post-translational modifications. Cloning vectors are available that increase the solubility of the expressed polypeptide. See, for example, U.S. Pat. Nos. 7,501,484 and 7,524,648.

As used herein, the term “host” or “host cell” is meant to include not only prokaryotes, such as E. coli, but also eukaryotes, such as yeast, insect, plant and animal cells. Animal cells include, for example, COS cells and HeLa cells. A host cell can be transformed or transfected with a DNA molecule (e.g., a vector or construct) using any of the techniques commonly known to those of ordinary skill in this art, such as calcium phosphate or lithium acetate precipitation, electroporation, lipofection and particle bombardment. Host cells containing a vector as described herein may be used for purposes such as propagating the vector, producing peripherin nucleic acid (e.g., DNA, RNA, antisense RNA), or expressing a peripherin polypeptide or fragments thereof.

Methods of producing peripherin polypeptides from peripherin nucleic acids are provided. Methods of producing peripherin polypeptides include, but are not limited to, culturing host cells containing a peripherin expression vector under conditions permissive for expression of peripherin, and recovering (e.g., purifying) the peripherin polypeptides. Methods of culturing bacteria and recovering expressed polypeptides, including insoluble polypeptides, are well known to those of ordinary skill in this art.

### Methods of Treatment

Further provided by this disclosure are methods of treating an individual whose immune system is producing peripherin-specific autoantibodies. Treatment of such a peripherin-associated autoimmune disease can include, without limitation, apheresis and/or T cell receptor-based immunotherapy.

Methods and extracorporeal systems for apheresis (i.e., the process of withdrawing blood from an individual, removing components from the blood, and returning the blood, or blood depleted of one or more components, to the individual) are known in the art (see, for example, U.S. Pat. Nos. 4,708,713; 5,258,503; 5,386,734; and 6,409,696). This disclosure provides a method of removing peripherin-specific autoantibodies from a body fluid of an individual. The method involves withdrawing a body fluid from a subject; removing a substantial portion of peripherin-specific autoantibodies from the fluid; and returning the fluid to the subject. Antibodies removed can be of any class, e.g., IgG (such as IgG1, IgG2, IgG3, IgG4), IgM, IgD, IgA, or IgE antibodies.

As used herein, a “substantial portion” means removing at least 20% (e.g., at least: 20%; 30%; 40%; 50%; 60%; 65%; 70%; 75%; 80%; 85%; 90%; 93%; 95%; 96%; 97%; 98%; 99%; 99.5%; 99.8%; or even 100%) of the peripherin-specific autoantibodies that were present in the body fluid prior to removal. The body fluid can be blood plasma or any other body fluid, e.g., lymph or cerebrospinal fluid. According to the methods described herein, depleting peripherin-specific autoantibodies from individuals with a peripherin-associated autoimmune disease may result in a reduction or a decrease in one or more of the symptoms.

Removal of peripherin-specific autoantibodies is generally performed by contacting a body fluid with a peripherin polypeptide or fragment thereof. The peripherin polypeptide or fragment thereof can be bound to a solid support. Such solid supports can be, without limitation, membranes, fibers, spherical beads, or granules and can be made with a water-insoluble, preferably porous, biocompatible material, e.g., organic polymers such as agarose, dextran, and polyacrylamide, or inorganic porous materials such as porous glass or porous silica gel. Such materials are suitable or can be adapted (e.g., derivatized with appropriate chemical groups) for attachment of a peripherin polypeptide.

When the body fluid is blood, the plasma and/or white blood cells can be separated from red blood cells (e.g., erythrocytes) and the red blood cells can be returned to the individual with or without white blood cells. Usually, the blood cells are returned to the individual with artificial rather than their original blood plasma. The “replacement fluid” (e.g., physiological saline) can be administered to the individual after removal of the fluid. Alternatively, the peripherin-specific autoantibodies can be selectively removed from the blood plasma in the course of apheresis and the blood cells can be mixed with the peripherin-specific autoantibody-depleted plasma and then re-infused as a mixture into the individual.

The system can be a continuous one in which, for example, blood is pumped out of a blood vessel (e.g., an artery or a vein) passed over a solid support derivatized with peripherin polypeptides and pumped directly back into a blood vessel of the subject. As in non-continuous systems, blood cells can be separated from plasma prior to passing of the plasma over the solid support.

In addition to apheresis, methods of T cell receptor therapy are known in the art. See, for example, Fujio et al., 2007, Ann. N.Y. Acad. Sci., 1110:222-32; Offner et al., 2008, Rev. Neurosci., 19:327-39; and Bendle et al., 2009, Curr. Opin. Immunol., 21:209-14. Monoclonal or polyclonal antibodies having specific binding affinity for the antigen(s) expressed by the peripherin-receptor or other marker on the T cell population responsible for inducing and maintaining the production of peripherin-specific autoantibodies can be used to deplete or suppress one or more pathogenic T cells. CDR3 spectratyping of T cell receptors can be used to identify autoimmune disease-associated T cell receptors (Matsumoto et al., supra; and Jambou et al., 2003, J. Clin. Invest., 112:254-74). In addition, activation of T cells can be inhibited in an individual by administering a cytokine or an antibody having specific binding affinity for a cytokine For example, to decrease a Th1-type immune response, a cytokine such as interleukin (IL)-4, IL-10, or IL-13, or an antibody specific for a cytokine such as IL-12 or IFN-γ can be administered to an individual. Similarly, to inhibit a Th2-type immune response, a cytokine such as IL-12 or IFN-γ or an antibody specific for IL-4, IL-10, or IL-13 can be administered to an individual.

Also provided are methods of enumerating or isolating peripherin-specific T-cells from an individual. This method may be used, for example, to monitor an individual's immune response or for immunotherapy using peripherin-specific cytotoxic T-cells. The method comprises contacting a biological sample containing lymphocytes with tetrameric soluble class I or class II major histocompatibility complex (MHC) bearing identical peripherin polypeptides or fragments thereof. Linker molecules such as avidin and biotin are used to produce the peripherin polypeptide-MHC tetrameric complex, which can subsequently be labeled with an indicator molecule such that those T-cells that recognize the peripherin polypeptide-MHC tetrameric complex are enumerated or isolated (e.g., using FACS analysis). See, for example, Schwartz, 1998, New England J. Med., 339:1076-8, and references therein.

In addition to apheresis and T-cell receptor therapy, therapeutic methods can include administering an effective amount of a pharmaceutical composition (e.g., a peripherin polypeptide or a nucleic acid such as an antisense oligonucleotide or a nucleic acid encoding a peripherin polypeptide) to an individual. An effective amount is an amount that deviates the individual's peripherin-mediated immune response, thereby modulating the autoimmune disease in the individual. As used herein, “modulating” an autoimmune disease can refer to reducing the severity of one or more symptoms, eliminating all symptoms, or any level of symptoms therebetween.

Peripherin polypeptides can be delivered to an individual directly or by administering a vector appropriately expressing a nucleic acid encoding a peripherin polypeptide. Vectors for delivering nucleic acids that encode biologically useful proteins to an individual are known in the art and include, for example, adenovirus, adeno-associated virus, retroviruses, lentiviruses, vaccinia virus, herpes viruses, and bovine papilloma virus nucleic acids. As used herein, “administering” refers to a method of delivering a composition (e.g., a peripherin polypeptide, a nucleic acid encoding a peripherin polypeptide, or an antisense oligonucleotide that hybridizes specifically to a portion of the nucleic acid encoding a peripherin polypeptide) to an individual. Routes of administration include, but are not limited to, oral, nasal, intravenous, intramuscular, intraperitoneal, subcutaneous, intrathecal, intradermal, or topical administration. The amount of a composition administered will depend on many factors, including the route of administration and the formulation (e.g., solid, liquid, or emulsion). Methods for formulating and subsequently administering therapeutic compositions are well known to those skilled in the art. See, for example, Remington, 2000, The Science and Practice of Pharmacy, 20th Ed., Gennaro & Gennaro, eds., Lippincott, Williams & Wilkins.

Also provided is a method of imaging peripherin polypeptide-expressing cells in an individual. The method comprises administering to the individual an effective amount of an anti-peripherin antibody having specific binding affinity for a peripherin polypeptide labeled with an imaging agent, for example, ³²P, ⁹⁹Tc, ¹¹¹In or ¹³¹I, to bind to a peripherin polypeptide released from, or accessible in, cells, and detecting any complex so formed. As is well known to those of ordinary skill in the art, a suitable amount of an anti-peripherin antibody is any amount that is effective to image cells, for example, about 0.1 mCi to about 50.0 mCi. In addition, an effective amount of an anti-peripherin antibody may be an amount from about 0.01 mg to about 100 mg. Suitable methods of administering the imaging agent are as described above and can be targeted (e.g., to the brain) as described above. Methods of imaging are dependent upon the agent used and are well known to those of skill in this art.

Representative materials and methods are described in the following examples, which do not limit the scope of the invention described in the claims.

### EXAMPLES

### Example 1

### Prospective Screening

The study was approved by the Mayo Foundation Institutional Review Board (IRB#06-007020). Between Jan. 1, 1998 and Dec. 31, 2008, Mayo Clinic's Neuroimmunology Laboratory prospectively screened sera from approximately 160,000 individuals for evidence of neurological autoimmunity. In addition to immunoprecipitation assays for cation channel autoantibodies, the sera were screened by indirect immunofluorescence for IgG binding selectively to neural elements in a composite substrate of mouse brain, gut and kidney tissues (Pittock et al., 2004, Mayo Clin. Proc., 81:1207-14). In some cases, an IgG was detected that bound to discrete filamentous elements in the central and peripheral nervous system. Review of clinical correlations in the first 5 individuals suggested a significant association with autonomic dysfunction, particularly gastrointestinal dysmotility. To define the novel autoantibody's clinical accompaniments more rigorously, an additional 21 individuals were prospectively collected whose sera yielded this immunostaining pattern and for whom adequate clinical information was available. Records of seropositive individuals were reviewed, and pertinent history, physical examination findings and laboratory reports (imaging, electrophysiological, physiological [autonomic reflex screen and thermoregulatory sweat test] and autoimmune serology) were extracted and tabulated.

### Example 2

### Rat Cell Lines

Cells were plated on poly-L-lysine-coated glass coverslips (BD BioCoat 354085) and held at 37° C. in a humidified atmosphere of 5-7% CO₂/95-93% air. Culture media were supplemented with 1 mM L-glutamine (Sigma-Aldrich G8540-100G) and penicillin/streptomycin (Invitrogen 15140-163). PC12 pheochromocytoma cells (ATCC CRL-1721) were maintained in DMEM 4.5 (Invitrogen 12100-061) supplemented with 10% fetal calf serum (Atlas Biologicals, Cat No. F-0500-A) and 10% horse serum (HyClone Labs, Cat No. A-3311-L). To promote differentiation, mouse nerve growth factor (2.5S; Alamone Laboratory, Cat No. N-100) was added on alternate days for 7 days (100 ng/mL). L6 skeletal muscle cells (ATCC, CRL-1458) were maintained in DMEM 4.5 supplemented with 10% horse serum. To promote differentiation, 2% fetal calf serum was substituted for 3 days; myotube formation was confirmed by microscopic examination. CG4 oligodendroglial-astrocytic progenitor cells (provided by Dr. Charles Howe, Department of Neurology, Mayo Clinic, Rochester, Minn.) were grown in proliferation medium and differentiated as described previously (Hinson et al., 2008, J. Exp. Med., 205:2473-81).

### Example 3

### Antibody Probes

Rabbit anti-neurofilament M (Chemicon AB1987), chicken anti-GFAP-Cy3 conjugated (Sigma C9205), rabbit anti-peripherin (Chemicon AB1530), mouse anti-peripherin (Chemicon MAB1527), goat anti-PDX-1 (Abcam ab47383) and human serum containing IgG reactive with skeletal muscle contractile proteins (myasthenia gravis individual, 83-4868) were obtained as indicated. Species-specific anti-IgG antibodies, conjugated to fluorochrome or horseradish peroxidase, were obtained from Southern Biotechnology Associates, Inc.

### Example 4

### Immunofluorescence

Individuals' sera were initially diluted (1:240) in PBS containing 1% BSA, and pre-absorbed with beef liver powder. The clinical substrate, a composite frozen section (4-μm-thick) of 3 tissues (adult mouse cerebellum/midbrain, gut and kidney) was post-fixed with 10% formalin. Cell substrates, attached to coverslips, were washed in PBS, fixed in 95% ethanol/5% acetic acid (−20° C., 15 minutes), rinsed in PBS, and permeabilized by exposing for 3 minutes to 0.05% Triton X100. All substrates were blocked with normal goat serum (10% in PBS; 15 minutes). Commercial antibody probes were diluted in block buffer. After applying primary antibodies (45 minutes), and washing, species-appropriate secondary antibodies were applied (45 minutes), substrates were rinsed, ProLong anti-fade medium (Molecular Probes P36935) was applied and coverslips were mounted. Individual sera yielding positive results were titrated in doubling dilutions to determine endpoints of antibody detection.

### Example 5

### Multi-Antigen Labeling

Tissues from a female mouse (aged 6-8 weeks) were oriented in OCT and snap frozen in isopentane. Cryosections (8 μm) were air-dried, incubated sequentially in PBS containing 10% normal goat serum or 1% BSA (30 minutes), primary antibody (45 minutes) and secondary antibody (45 minutes). After washing, they were mounted in ProLong anti-fade agent containing DAPI and analyzed by confocal microscopy. Animal studies were approved by the Mayo Institutional Animal Use and Care Committee (A36207).

### Example 6

### Protein Extraction and Western Blotting

PC12 cells (100 μL packed volume) were extracted in 2 mL buffer (0.15 M NaCl, 0.01 M NaPO₄, 2 mM EDTA, pH 7.2) containing 1% NP40, 0.1% SDS and protease inhibitors (Complete™, Roche 11697498001). After shaking (4° C., 1 hour), proteins were denatured and reduced by boiling (5 minutes) in 2% SDS and 2-mercaptoethanol, separated by electrophoresis in 10% polyacrylamide gel, and transferred to nitrocellulose for Western blot. Molecular weight standards included biotinylated broad range markers (BioRad 161-0322) or pre-stained SDS-PAGE standards (BioRad 161-0374).

### Example 7

### Affinity Purification of Individual IgG

PC12 lysate proteins were transblotted to nitrocellulose. Bound antigenic protein was located by Western blot staining of excised vertical edge strips. The horizontal intervening strip bearing antigen, and a control horizontal strip lacking the antigen of interest, were probed with individual serum. Both were washed three times in high salt buffer (50 mM Tris-HCl, pH 7.4 containing 500 mM NaCl), and once in low salt buffer (100 mM NaCl). Bound IgG was eluted in glacial acetic acid, neutralized, dialyzed against PBS, 0.02% sodium azide and applied to the composite mouse tissue substrate to evaluate the immunofluorescence staining pattern.

### Example 8

### Sub-Cellular Fractionation

Four fractions (cytosol, membrane, nucleus and cytoskeleton) were prepared from PC12 cells using a Subcellular Proteome Extraction kit.(Calbiochem 539790). After electrophoresis in denaturing 10% polyacrylamide gel, proteins were transblotted to nitrocellulose and probed with positive individual serum.

### Example 9

### Two-Dimensional (2D) Gel Electrophoresis, In-Gel Trypsin Digestion and Mass Spectrometry

The fraction containing the putative antigen was separated by 2D gel electrophoresis using published methods (12). Proteins were visualized by silver staining and immunoblotting. Prominent antigenic spots were excised and subjected to in-gel digestion and analysis by tandem mass spectrometry (Jimenez et al., 1998, Current Protocols in Protein Science, S14, John Wiley & Sons, Inc.). Peptides were identified using the MASCOT search algorithm (Kapp et al., 2007, Current Protocols in Protein Science, S49, John Wiley & Sons, Inc.).

### Example 10

### Clinical-Serological Correlations of a Novel Neural Autoantibody

A total of 26 seropositive individuals were initially identified for whom clinical records were available (16 women, 10 men). The novel autoantibody was not detected in any of 173 age-matched healthy control subjects. Table 1 summarizes clinical and laboratory information and serological data for the individuals. The median age of the seropositive individuals was 46 years (range 21-86); and the median titer of the novel IgG was 3,840 (range, 240 to 30,720). All but 8 were evaluated at Mayo Clinic. Twenty-five had neurological complaints; and 19 (73%) had dysautonomia or endocrinopathy.

Diverse neurological manifestations were documented in individuals: sensorimotor neuropathy, lumbosacral plexopathy, transverse myelitis, non-specified myelopathy, optic neuropathy, encephalitis, cerebellar ataxia and myasthenia gravis. Six individuals (23%) had one or more co-existing neural autoantibodies (voltage-gated neuronal calcium channel or potassium channel, GAD 65 [3 individuals], ganglionic acetylcholine receptor, CRMP-5).

Dysautonomia was the most commonly documented clinical association (14 individuals; 54%). It was generalized in 2 and limited in 12; 8 had gastrointestinal (GI) dysmotility and 6 had abnormalities of sudomotor, cardiovagal or adrenergic functions. GI dysmotility was confirmed by endoscopy, manometry or transit studies.

Nine individuals (35%) had a clinically documented endocrinopathy, a marker autoantibody of endocrine autoimmunity or both. Endocrinologic disorders, documented in 7 individuals, included diabetes (two individuals had autoantibodies specific for both GAD65 and islet antigen-2 [IA-2]), thyroid disorders, and premature menopause. Two individuals had thyroid autoantibodies (thyroperoxidase [2] or thyroglobulin [1]) without documented thyroid dysfunction.

In continuing experiments, the peripherin-IgG increasingly is recognized as a biomarker for a subset of patients with inflammatory autoimmune CNS disorders that mimic multiple sclerosis (as epitomized by 3 of the original patients; numbers 11, 15 and 26 in Table 1). Nine patients with the “MS mimic phenotype” are described in Table 2.

TABLE 1
Clinical-serological correlations in seropositive individuals

| | | peri- | | | | | |
|---|---|---|---|---|---|---|---|
| | | pherin | Signs of dysautonomia | | | | |
| | Age* | IgG | | Limited | | | |
| | (yrs)/sex/ | Titer, | | Gastro- | | Other neurological | |
| Patient # | race | IF | Generalized | intestinal | Other^{†} | symptoms or signs | Endocrinopathy |
| 1^{‡} | 79/M/U | 240 | − | Achalasia, | — | — | — |
| | | | | gastroparesis, | | | |
| | | | | weight loss | | | |
| 2^{‡} | 60/F/C | 240 | − | Achalasia, | — | — | — |
| | | | | weight loss | | | |
| 3^{‡} | 38/F/C | 1,920 | − | Dysphagia, | — | — | — |
| | | | | weight loss | | | |
| 4 | 26/F/AA | 1,920 | + | Gastroparesis, | ART abnormal | — | Diabetes |
| | | | | delayed bowel | | | |
| | | | | transit | | | |
| 5^{‡} | 46/F/C | 3,840 | − | — | Orthostatic | Numbness in feet, | — |
| | | | | | hypotension | urinary urgency, | |
| | | | | | (ART and TST | incontinence | |
| | | | | | abnormal) | | |
| 6^{‡} | 39/F/C | 240 | − | Delayed | — | — | — |
| | | | | gastrointestinal | | | |
| | | | | transit | | | |
| 7^{‡,§} | 70/M/C | 480 | − | — | Orthostatic | Right homonymous | — |
| | | | | | hypotension | hemianopia due to | |
| | | | | | with syncope | occipital | |
| | | | | | (ART abnormal) | metastasis^{§} | |
| 8^{‡} | 35/F/U | 3840 | − | — | ART consistent | Fatigue, diffuse | — |
| | | | | | with POTS | muscle stiffness, | |
| | | | | | (postural | numbness both | |
| | | | | | orthostatic | hands and feet; | |
| | | | | | tachycardia | fibromyalgia | |
| | | | | | syndrome) | | |
| 9^{‡} | 43/F/C | 3,840 | − | — | — (ART | Numbness, tingling, | Hypothyroid, |
| | | | | | normal) | painful feet and | premature |
| | | | | | | hands | menopause |
| 10^{‡} | 40/F/U | 1,920 | − | — | — | Sensorimotor | Hypothyroid; |
| | | | | | | neuropathy | premature |
| | | | | | | | menopause |
| 11 | 43/F/U | 3,840 | − | — | — | Optic neuropathy | — |
| 12^{‡} | 39/F/C | 960 | − | Delayed small | — (ART | Dizziness, | — |
| | | | | bowel motility | normal) | palpitations | |
| | | | | transit | | | |
| 13^{‡,§} | 70/F/C | 1,920 | − | — (Weight | Mild orthostatic | Left leg weakness, | Hyperthyroid, |
| | | | | loss only) | dizziness (ART | pain; left | diabetes |
| | | | | | and TST | lumbosacral | |
| | | | | | abnormal) | plexopathy | |
| 14^{‡} | 45/M/C | 7,680 | − | — | — | — | Diabetes |
| 15^{‡} | 47/M/C | 1,920 | − | — | — | Transverse myelitis | Hypothyroid |
| | | | | | | (spinal cord | |
| | | | | | | enhancing lesion at | |
| | | | | | | C5) level in spinal | |
| | | | | | | cord) | |
| 16^{‡} | 46/F/C | 3,840 | − | — | — | Neck pain | — |
| 17^{‡} | 66/M/U | 3,840 | − | — | — | Myasthenia gravis | Diabetes, |
| | | | | | | | hypothyroidism |
| 18^{‡} | 47/F/C | 960 | − | Achalasia, | — | — | — |
| | | | | weight loss | | | |
| 19 | 27/M/C | 30,720 | − | — | Mild | Polyradiculoneuropathy | — |
| | | | | | orthostatism | (improvement | |
| | | | | | | followed with IVIG | |
| | | | | | | therapy) | |
| 20 | 59/M/C | 7,680 | − | — | — | Postural orthostatic | — |
| | | | | | | tremor | |
| 21 | 47/M/C | 7,680 | + | Dysphagia, | ART: small | Erectile dysfunction | — |
| | | | | nausea, | fiber neuropathy | | |
| | | | | vomiting, | | | |
| | | | | weight loss | | | |
| 22 | 86/M/C | 15,360 | − | — | — | Cerebellar ataxia | — |
| 23^{§} | 52/F/C | 30,720 | − | — | Hypotension, | — | — |
| | | | | | anxiety | | |
| 24 | 40/F/H | 7,680 | − | — | — | Encephalitis with | — |
| | | | | | | seizures | |
| 25^{‡} | 21/M/C | 3,840 | − | — | — | Generalized body | — |
| | | | | | | pain, opisthotonic | |
| | | | | | | spasms; no | |
| | | | | | | diagnosis despite | |
| | | | | | | extensive | |
| | | | | | | evaluation | |
| 26 | 60/F/C | 30,720 | − | — | — | Myelopathy | — |

*Age at autoantibody detection; Sex: M, male; F, female; Ethnicities: C, Caucasian; AA, African American; H, Hispanic; U, unknown.
^{†}8 patients underwent autonomic laboratory evaluation of postganglionic sudomotor, cardiovagal, and adrenergic functions (ART, Autonomic Reflex Test; TST, Thermoregulatory Sweat Test).
^{‡}Mayo Clinic Patients.
^{§}Cancer detected: Patient 7, adenosquamous cell lung carcinoma metastatic to left occipital lobe 8 years before autoantibody detection; Patient 13, pituitary adenoma (panhypopituitarism followed resection); Patient 23, breast carcinoma. Eight patients had a coexisting organ-specific autoantibody or disease: voltage-gated calcium channel, N-type (patient 4); voltage-gated potassium channel (patient 24); glutamic acid decarboxylase-65 (patients 4, 14 and 24); IA-2 islet cell antigen (patients 4 and 14); muscle acetylcholine receptor (patient 17); ganglionic acetylcholine receptor (patient 19); thyroblobulin (patients 5, 14 and 24); thyroid peroxidase (patient 5); collapsin response-mediator protein(CRMP)-5 (patient 17); psoriasis (patient 18); Sjogren syndrome (patient 9).

TABLE 2
Peripherin-IgG a biomarker for a subset of inflammatory autoimmune CNS
disorders presenting with predominant involvement of anterior visual pathways (optic
nerve) and spinal cord (multiple sclerosis mimics), with and without endocrinopathy (all
confirmed by Western blot)

| | | Age at | Peripherin- | | | | | |
|---|---|---|---|---|---|---|---|---|
| | | CNS | IgG titer | | | | | |
| | | symptom | (immuno- | Findings | | | | |
| Patient # | Sex | onset | fluorescence)* | Spinal cord | Optic nerve | Brain | Other | Endocrinopathy |
| 1 | M | 47 | >480 | Myelopathy- | None | MRI: | Cerebellar | Type 1 DM, |
| | | | | anterior horn cell | | corticospinal | ataxia, | hypothyroidism |
| | | | | involvement | | tract signal | peripheral | |
| | | | | | | abnormality | neuropathy, | |
| | | | | | | | erectile | |
| | | | | | | | dysfunction | |
| 2 | F | 16 | >480 | Normal | Bilateral optic | Normal MRI | — | — |
| | | | | | neuritis; | | | |
| | | | | | steroid- | | | |
| | | | | | responsive | | | |
| 3 | F | 39 | 960 | Progressive | — | MRI: | — | — |
| | | | | myelopathy | | demyelination | | |
| 4 | M | 33 | 960 | Myelopathy | Unilateral | MRI: | — | — |
| | | | | | optic neuritis, | demyelination | | |
| | | | | | episodes | | | |
| | | | | | affecting left | | | |
| | | | | | and right eye | | | |
| 5 | F | 38 | 1920 | Myelopathy-MRI | Recurrent | Normal MRI | — | hyperthyroid |
| | | | | normal | unilateral | | | |
| | | | | | optic neuritis; | | | |
| | | | | | steroid- | | | |
| | | | | | responsive | | | |
| 6 | F | 35 | >480 | Myelopathy- | — | Normal MRI | — | — |
| | | | | sensory only- | | | | |
| | | | | MRI | | | | |
| | | | | longitudinally | | | | |
| | | | | extensive lesion | | | | |
| | | | | C2-C8 | | | | |
| 7 | M | 74 | 32 (CSF) | TM-paraplegia- | | MRI: | — | DM, |
| | | | | MRI shows | | demyelination | | hypothyroidism |
| | | | | longitudinally | | | | |
| | | | | extensive lesion | | | | |
| | | | | T9-conus | | | | |
| 8 | F | 28 | 15,360 | TM-paraplegia- | — | Normal MRI | — | — |
| | | | | MRI shows | | | | |
| | | | | longitudinally | | | | |
| | | | | extensive lesion- | | | | |
| | | | | Herpes simplex | | | | |
| | | | | virus PCR | | | | |
| | | | | positive in CSF; | | | | |
| | | | | improved with | | | | |
| | | | | acyclovir | | | | |
| 9 | F | 39 | 61,440 | TM→progressive | Optical | MRI: | — | — |
| | | | | myelopathy- | coherence | demyelination | | |
| | | | | MRI shows | tomography: | | | |
| | | | | lesions cervical | retinal nerve | | | |
| | | | | (upper and | fiber thinning, | | | |
| | | | | lower) and | right | | | |
| | | | | thoracic (T6-T8) | | | | |
| | | | | cord | | | | |

### Example 11

### Immunohistochemical Characteristics

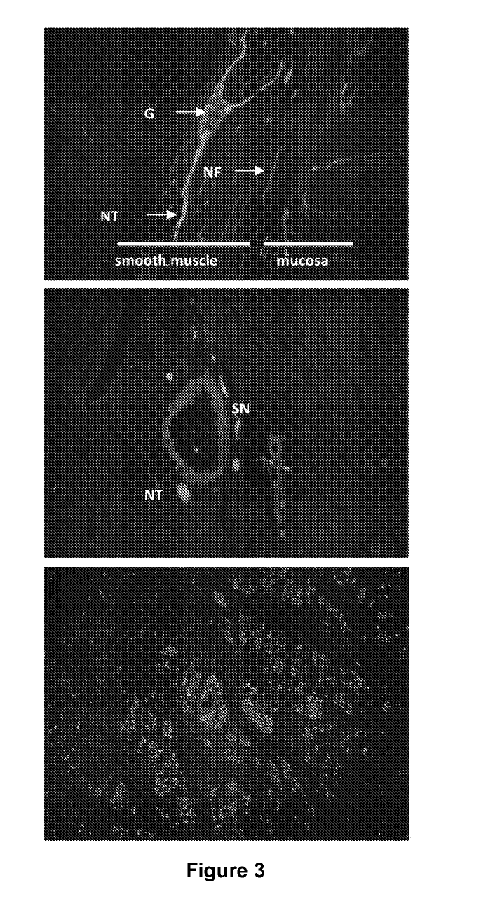

FIG. 3 illustrates the distinctive pattern of human IgG immunostaining on mouse tissues. In stomach (upper panel), immunoreactive elements were prominent in neural elements of smooth muscle (enteric ganglia and nerve trunks), mucosa and submucosa (nerve fibers). In kidney (middle panel), immunoreactivity was restricted to sympathetic nerve trunks and fibers near arteries and arterioles. The mid-hind brain (lower panel) contained discrete immunoreactive nerve tracts.

### Example 12

### Neurons Contain Abundant Autoantigen

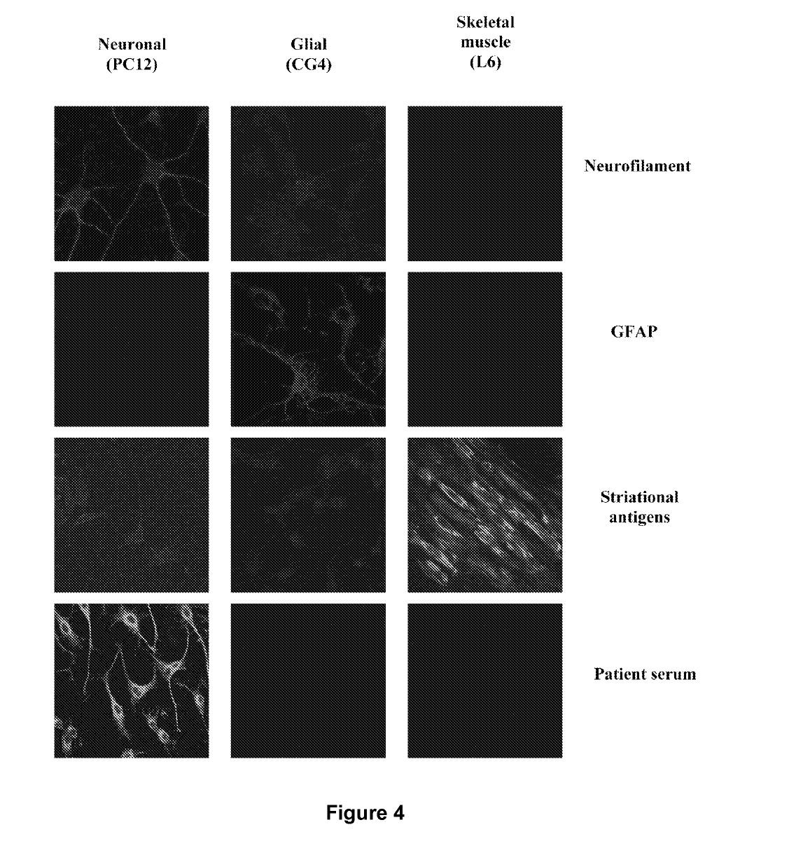

To unambiguously identify the cell types expressing immunoreactivity, a panel of rat cell lines was investigated using individual IgG and defined IgG probes that detected cell-type appropriate filaments (FIG. 4) in PC12 pheochromocytoma cells, CG4 glial cells and L6 skeletal muscle cells (respectively, neurofilaments, GFAP intermediate filaments and sarcomeric striational antigens (Williams et al., 1991, Clin. Immunol. Newsletter, 11:161-170). The novel human autoantibody bound to filaments that were restricted to the cytoplasm of PC12 neuronal cells. PC12 neuronal cells were used thereafter as the source of autoantigen for immunochemical and molecular analyses.

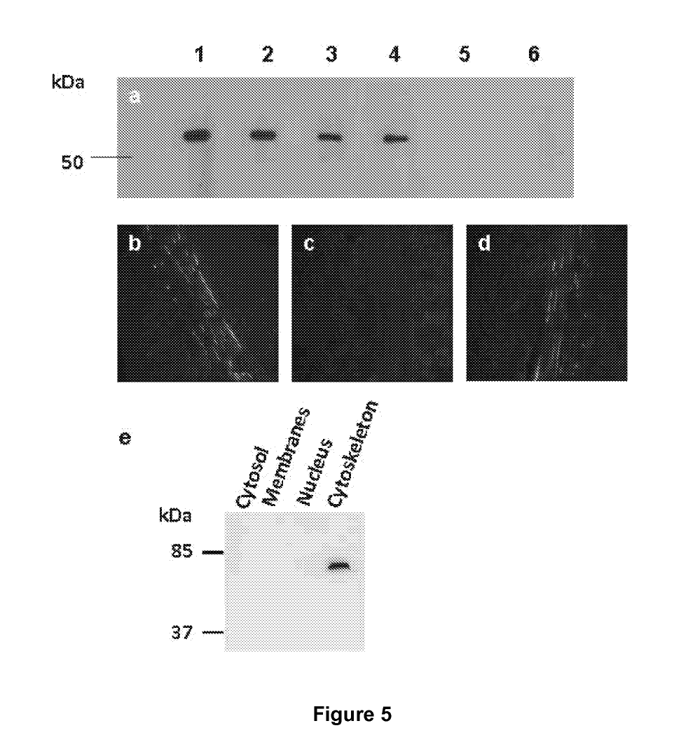

Western blot defined an IgG in individual sera (18 of 18 tested) that bound to a PC12 lysate protein of Mr ˜55 kDa (FIG. 5A). To verify that this protein represented the antigen defined by immunostaining, electrophoretically-separated proteins were transferred to nitrocellulose and edge strips were probed with individual IgG to identify the immunoreactive band. The horizontal strip containing immunoreactivity, plus an irrelevant strip containing lower molecular weight proteins, were excised and exposed to individual serum or control human serum. After washing extensively, bound IgGs were eluted and applied to the mouse triple tissue substrate. IgG eluted from the ˜55kDa protein strip, but not from the irrelevant protein strip, yielded the characteristic staining pattern observed with the individual's original serum (FIG. 5B-D).

### Example 13

### Identification of the Neuronal Autoantigen

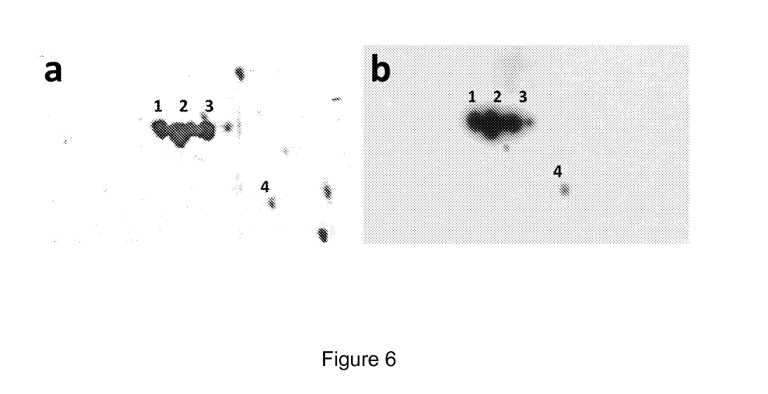

Western blot analysis of PC12 lysate fractions enriched for cytosol, membrane, nucleus or cytoskeleton demonstrated that IgG in the individual sera bound exclusively to a cytoskeletal protein (FIG. 5E). Initial attempts to purify the antigen were hampered by its insolubility, but a combination of 2D electrophoresis, immunoblotting and mass spectrometry analyses resulted in purified antigen. Silver staining and immunoblotting both revealed 4 identical spots (FIG. 6). Analysis of each spot by in-gel digestion and mass spectrometry yielded 41, 47, 38 and 36 unique peptides that contained peripherin-unique sequences.

### Example 14

### Peripherin is Expressed in Multiple Endocrine Tissues

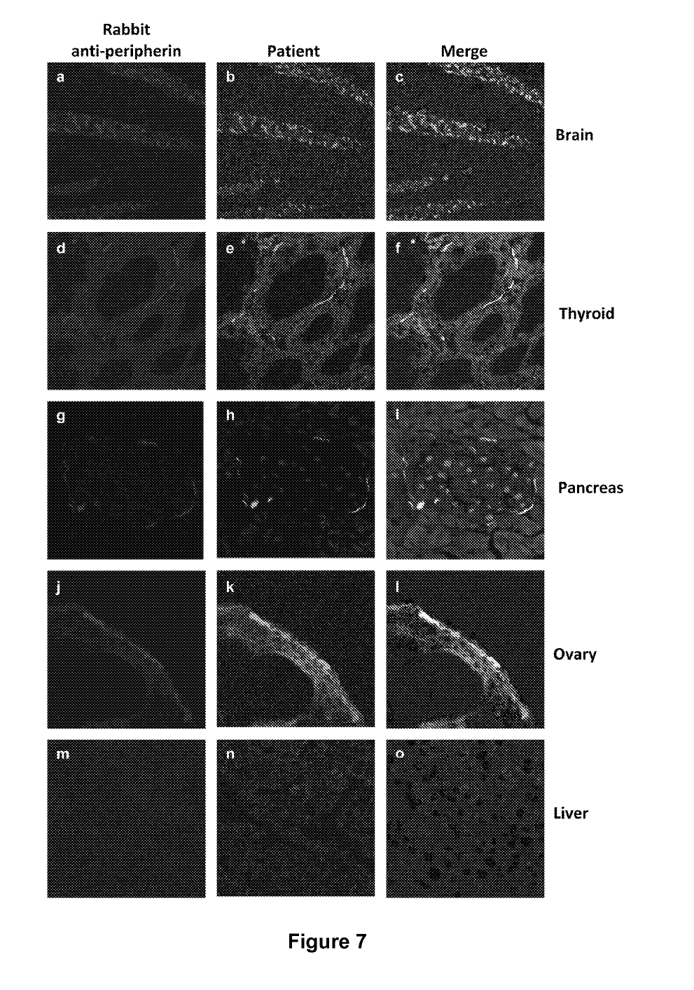

Rabbit anti-peripherin IgG, individual sera and adult mouse tissues were used to investigate the distribution of peripherin in the endocrine tissues for which 35% of the peripherin-IgG-positive individuals in this report had clinical or serological evidence of autoimmunity, namely thyroid, pancreas and ovary. Brain served as a positive control tissue, and liver as a negative control tissue (McLean et al., 2008, J. Neurochem., 104:1663-73). The autoantigen defined in brain by individual IgG co-localized with peripherin (FIG. 7). Immunoreactivity was prominent in nerve fibers surrounding islets of Langerhans in the pancreas, nerve fibers in interstitial tissue between thyroid follicles, and in nerve fibers adjacent to ovarian follicles. Immunoreactivity was not detected in liver.

### Example 15

### Clinical Association of Peripherin-Specific Autoantibody

To estimate the frequency of peripherin-specific autoantibody among patients in whom seropositivity was incidentally detected within the clinically relevant diagnostic categories (Table 1), stored patient sera from the following clinically relevant diagnostic groups were selected for immunofluorescence and Western blot (as described above using PC12 cell lysate).

Group 1. Type 1 diabetes: n=28 (median age 20, age range 3-65);

Group 2. Various combinations of type 1 diabetes, premature menopause, thyroiditis, thymoma, pituitary tumor: n=9; and

Group 3. Small fiber peripheral neuropathy with/without autonomic neuropathy, premature menopause or type 1 diabetes: n=12.

None of the patients in any of the three groups were positive by IF. None of the patients in either of Group 1 or 2 were positive by Western blot, while four of the patients in Group 3 (33%) were positive by Western blot. These results are summarized in Table 3.

TABLE 3
Frequency of peripherin-IgG detection in archival sera of patient groups
selected by clinical diagnostic categories

| | | | | | | Peripherin-IgG | |
|---|---|---|---|---|---|---|---|
| | Number of | | | | | positive (%) | |
| | patients | | | Age, years^{a} | | Immuno | Western |
| Diagnostic group | F | M | Total | Median | Range | fluorescence | blot |
| Diabetes, type 1, no | 18 | 10 | 28 | 20 | 3-65 | 0 | 0 |
| neurological disorder | | | | | | | |
| Thymoma (7) or meningioma | 8 | 1 | 9 | 54 | 19-75 | 0 | 0 |
| (1) with or without 1 or more | | | | | | | |
| endocrinopathies (type 1 | | | | | | | |
| diabetes, premature | | | | | | | |
| menopause or thyroiditis) | | | | | | | |
| Small fiber neuropathy | 8 | 4 | 12 | 37 | 16-66 | 0 | 33 |
| with/without autonomic | | | | | | | |
| involvement, premature | | | | | | | |
| menopause or type 1 diabetes | | | | | | | |

^{a}age at blood draw

These findings suggest that the peripherin-specific autoantibody can be a diagnostically useful marker of small fiber peripheral neuropathy/autonomic neuropathy in patients with type I diabetes and/or thyroid disease, and may be predictive for development of small fiber peripheral neuropathy in patients with endocrine autoimmunity.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of detecting the presence or absence of a peripherin-specific autoantibody in a biological sample from an individual, comprising the steps of:
contacting said biological sample with a peripherin polypeptide or fragment thereof; and
detecting the presence or absence of binding of said peripherin polypeptide or fragment thereof to said peripherin-specific autoantibody in said biological sample.

2. The method of claim 1, wherein the presence of said peripherin-specific autoantibody in said biological sample is associated with dysautonomia or small fiber neuropathy, endocrinopathy, or one or more other neurological manifestations in said individual.

3. The method of claim 2, wherein the dysautonomia is chosen from gastrointestinal (GI) dysmotility, abnormal sudomotor function, abnormal cardiovagal function, or abnormal adrenergic function.

4. The method of claim 2, wherein said endocrinopathy is chosen from diabetes, thyroid disorders, or premature menopause.

5. The method of claim 2, wherein said one or more neurological manifestations is chosen from transverse myelitis, non-specified myelopathy, sensorimotor neuropathy, optic and other cranial neuropathies, encephalitis, cerebellar ataxia, or myasthenia gravis.

6. The method of claim 1, wherein said peripherin polypeptide or fragment thereof is in a cell lysate.

7. The method of claim 1, wherein said peripherin polypeptide is in a solid tissue selected from the group consisting of brain, cranial nerves, kidney, stomach or other tissues containing peripheral nerve elements.

8. The method of claim 1, wherein said biological sample is selected from the group consisting of serum, plasma, cerebrospinal fluid, and blood.

9. An article of manufacture, comprising a peripherin polypeptide or fragment thereof and instructions for using said peripherin polypeptide to detect an anti-peripherin autoantibody in an individual.

10. The article of manufacture of claim 9, wherein said article of manufacture is used to diagnose the presence or absence of a peripherin-associated autoimmune disease in said individual.

11. The article of manufacture of claim 9, further comprising a monoclonal antibody having specific binding affinity for a peripherin polypeptide or fragment thereof.

12. A method of treating an individual having a peripherin-associated autoimmune disease, said method comprising:
withdrawing a body fluid from the individual, wherein the body fluid contains one or more autoantibodies that bind to peripherin;
removing a substantial portion of the autoantibodies from the body fluid; and
returning the body fluid to the subject.

13. A method of treating an individual having a peripherin-associated autoimmune disease, said method comprising:
administering a peripherin polypeptide to said individual.

14. The method of claim 13, wherein said administration is by a method selected from the group consisting of orally, intravenously, and parenterally.
